(19) United States
(12) Patent Application Publication
(10) Pub. No.: US 2007/0208405 A1
(43) Pub. Date: Sep. 6, 2007

(54) STENT DELIVERY CATHETER

(75) Inventors: Richard L. Goodin, Blaine, MN (US); Richard C. Gunderson, Maple Grove, MN (US); Clark C. Davis, West Valley City, UT (US); Todd H. Turnlund, Circle Park City, UT (US); John A. Lippert, Incline Village, NV (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/276,558

(22) Filed: Mar. 6, 2006

(51) Int. Cl. A61F 2/06

(52) U.S. Cl. 623/1.11

(57) ABSTRACT

Stent delivery catheters adapted to provide both flexibility and strength are disclosed. Such stent delivery catheters may have outer shafts adapted for tensile strength and inner shafts adapted for compressive strength. In some instances, at least one of the outer shaft and/or the inner shaft may include a micromachined portion.

### TECHNICAL FIELD

The invention relates generally to catheters and relates more particularly to catheters that are adapted for stent delivery.

### BACKGROUND

Medical devices such as catheters may be subject to a number of often conflicting performance requirements such as flexibility and strength. Catheters such as stent delivery catheters are expected to exhibit flexibility so that a patient's vasculature can be navigated sufficiently to access a treatment site. Stent delivery catheters, particularly catheters for delivering self-expanding stents, are also expected to exhibit tensile and/or compressive strength.

A need remains, therefore, for stent delivery catheters adapted to provide both flexibility and strength. A need remains for stent delivery catheters having outer sheaths adapted for tensile strength and inner shafts adapted for compressive strength.

### SUMMARY

The invention pertains generally to stent delivery catheters that are adapted to provide both flexibility and strength. The invention pertains generally to stent delivery catheters having outer sheaths adapted for tensile strength and inner shafts adapted for compressive strength.

Accordingly, an example embodiment of the invention can be found in a stent delivery catheter having an outer shaft defining an outer shaft lumen, the outer shaft extending from a proximal region of the catheter to a distal region of the catheter. A inner shaft is disposed within the outer shaft lumen. At least one of the outer shaft and the inner shaft include a micromachined portion thereof.

Another example embodiment of the invention can be found in a catheter having a micromachined hypotube that has a proximal end and a distal end, the micromachined hypotube extending distally to a distal end of the catheter. A proximal tube extends proximally from the proximal end of the micromachined hypotube. An inflatable balloon defining a balloon interior is disposed over the micromachined hypotube.

Another example embodiment of the invention can be found in a micromachined hypotube that includes an elongate cylindrical shaft having an interior surface and an exterior surface. A plurality of rings that are sized and configured for tensile strength are formed within the elongate cylindrical shaft. A plurality of beams that are sized and configured for flexibility are interspersed between adjacent rings. A plurality of voids defining the plurality of rings and the plurality of beams extend between the interior surface and the exterior surface.

The above summary of the present invention is not intended to describe each disclosed embodiment or every implementation of the present invention. The Figures, Detailed Description and Examples which follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a side elevation view of a self-expanding stent delivery catheter in accordance with an embodiment of the invention;

FIG. 2 is a diagrammatic longitudinal cross-section of a self-expanding stent delivery catheter in accordance with an embodiment of the invention;

FIG. 3 is a diagrammatic longitudinal cross-section of a self-expanding stent delivery catheter in accordance with an embodiment of the invention;

FIG. 4 is a diagrammatic longitudinal cross-section of a self-expanding stent delivery catheter in accordance with an embodiment of the invention;

FIG. 5 is a diagrammatic longitudinal cross-section of a self-expanding stent delivery catheter in accordance with an embodiment of the invention;

FIG. 6 is a diagrammatic longitudinal cross-section of a self-expanding stent delivery catheter in accordance with an embodiment of the invention;

FIG. 7 is a radial cross-section of a portion of a stent delivery catheter in accordance with an embodiment of the invention;

FIG. 8 is a radial cross-section of a portion of a stent delivery catheter in accordance with an embodiment of the invention;

FIG. 9 is a diagrammatic longitudinal cross-section of a self-expanding stent delivery catheter in accordance with an embodiment of the invention;

FIG. 10 is a diagrammatic longitudinal cross-section of a balloon-inflatable stent delivery catheter in accordance with an embodiment of the invention;

FIG. 11 illustrates a particular micromachining pattern in accordance with an embodiment of the invention; and

FIG. 12 illustrates a hypotube machined in accordance with the micromachining pattern of FIG. 11.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

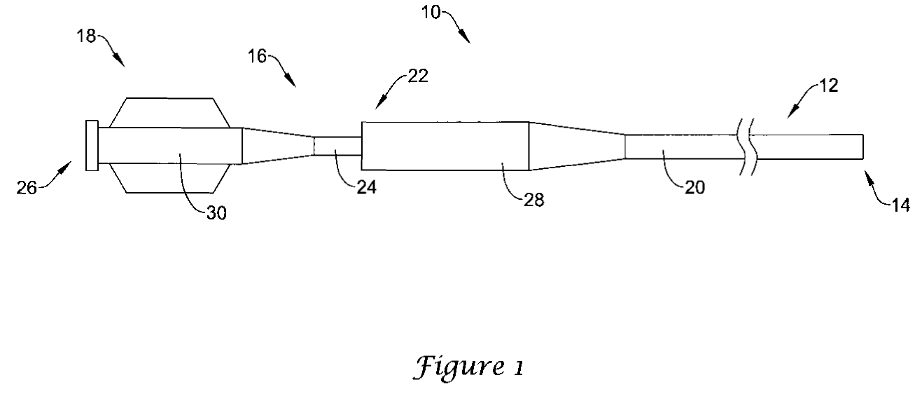

FIG. 1 is a plan view of a stent delivery catheter 10 in accordance with an embodiment of the present invention. As illustrated, stent delivery catheter 10 is adapted for delivering and deploying a self-expanding stent, although the invention is not limited to such. The catheter 10 can have a length that is in the range of about 100 to 150 centimeters and can have any useful diameter. Except as described herein, the stent delivery catheter 10 can be manufactured using conventional techniques.

In the illustrated embodiment, the stent delivery catheter 10 has a distal region 12 defining a distal end 14 and a proximal region 16 defining a proximal end 18. The stent delivery catheter 10 includes an elongate outer shaft 20 that itself has a proximal end 22 and a inner shaft 24 that itself includes a proximal end 26.

A hub and strain relief assembly 28 may be secured to the proximal end 22 of the outer shaft 20. In some embodiments, as illustrated, the inner shaft 24 may include a hub and strain relief assembly 30 secured to the proximal end 26 of the inner shaft 24. If present, the hub and strain relief 28 and the hub and strain relief 30 may be of conventional material and design, and may be attached using conventional techniques. It is also recognized that alternative hub designs can be incorporated into embodiments of the present invention.

The outer shaft 20 can include one or more shaft segments having varying degrees of flexibility. For example, the elongate shaft may include a relatively stiff proximal portion, a relatively flexible distal portion and an intermediate position disposed between the proximal and distal portions having a flexibility that is intermediate to both.

In some cases, the outer shaft 20 may be formed of a single polymeric layer, or from a plurality of polymeric layers. In some instances, the outer shaft 20 may include an inner liner such as an inner lubricious layer and an outer layer. In some cases, the outer shaft 20 may include a reinforcing braid layer disposed between the inner and outer layers. In some instances, the outer shaft 20 may include a reinforcing coil element that is disposed between the inner and outer layers, if present.

If the outer shaft 20 includes an inner liner, the inner liner can include or be formed from a coating of a material having a suitably low coefficient of friction. Examples of suitable materials include polytetrafluoroethylene (PTFE), better known as TEFLON®, and high density polyethylene (HDPE).

The outer shaft 20 can include, as an outer layer or layers, any suitable polymer that will provide the desired strength, flexibility or other desired characteristics. Polymers with low durometer or hardness can provide increased flexibility, while polymers with high durometer or hardness can provide increased stiffness. In some embodiments, the polymer material used is a thermoplastic polymer material. Some examples of some suitable materials include polyurethane, elastomeric polyamides, block polyamide/ethers (such as PEBAX®), silicones, and co-polymers.

The outer shaft 20 can be a single polymer, multiple longitudinal sections or layers, or a blend of polymers. By employing careful selection of materials and processing techniques, thermoplastic, solvent soluble, and thermosetting variants of these materials can be employed to achieve the desired results. In some instances, a thermoplastic polymer such as a co-polyester thermoplastic elastomer, for example, that available commercially under the ARNITEL® name, can be used.

In some instances, as will be discussed with respect to subsequent Figures, the outer shaft 20 may include or be formed from one or more metallic hypotubes. In some cases, outer shaft 20 may include or be formed from one or more micromachined hypotubes. The outer shaft 20 may include or be formed from a micromachined hypotube that is formed of any suitable material such as a metallic material including stainless steel or a nickel-titanium alloy such as Nitinol.

A micromachined hypotube may be formed having any desired length, width, material thickness, and slot size as required to satisfy the requirements of any particular application. Additional details concerning micromachined hypotube 10, including the manufacture thereof, can be found, for example, in U.S. Pat. No. 6,766,720 and published U.S. Patent Application No. 2004/0181174A2, each of which are fully incorporated, in their entirety, by reference herein.

The inner shaft 24 can include one or more shaft segments having varying degrees of flexibility. For example, the elongate shaft may include a relatively stiff proximal portion, a relatively flexible distal portion and an intermediate position disposed between the proximal and distal portions having a flexibility that is intermediate to both.

In some cases, the inner shaft 24 may be formed of a single polymeric layer, or of a plurality of polymeric layers. If the inner shaft 24 is formed of two or more polymeric layers, a reinforcing braid layer or a reinforcing coil element may be disposed between the polymeric layers. In some instances, the inner shaft 24 may include a liner formed of a lubricious material such as PTFE (polytetrafluoroethylene) or HDPE (high density polyethylene). In some instances, as will be discussed with respect to subsequent Figures, the inner shaft 24 may include or be formed from one or more metallic hypotubes.

The inner shaft 24 can include any suitable polymer that will provide the desired strength, flexibility or other desired characteristics. Polymers with low durometer or hardness can provide increased flexibility, while polymers with high durometer or hardness can provide increased stiffness. In some embodiments, the polymer material used is a thermoplastic polymer material. Some examples of some suitable materials include polyurethane, elastomeric polyamides, block polyamide/ethers (such as PEBAX®), silicones, and co-polymers.

The inner shaft 24 can be a single polymer, multiple longitudinal sections or layers, or a blend of polymers. By employing careful selection of materials and processing techniques, thermoplastic, solvent soluble, and thermosetting variants of these materials can be employed to achieve the desired results. In some instances, a thermoplastic polymer such as a co-polyester thermoplastic elastomer, for example, that available commercially under the ARNITEL® name, can be used.

In some instances, as will be discussed with respect to subsequent Figures, the inner shaft 24 may include or be formed from one or more metallic hypotubes. In some cases, inner shaft 24 may include or be formed from one or more micromachined hypotubes. The inner shaft 24 may include or be formed from a micromachined hypotube that is formed of any suitable material such as a metallic material including stainless steel or a nickel-titanium alloy such as Nitinol.

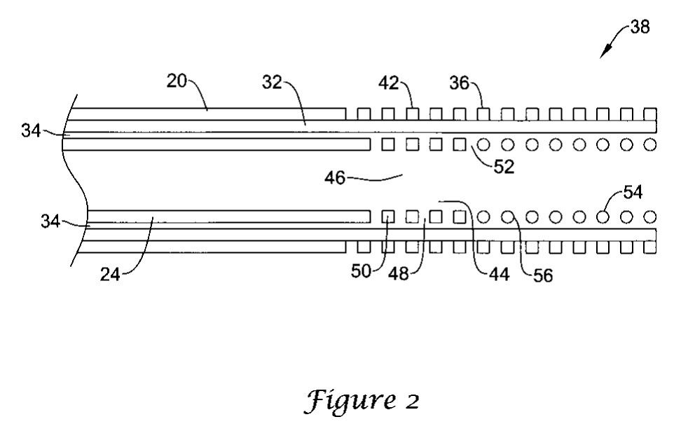

FIG. 2 illustrates a distal portion of the stent delivery catheter 10 (FIG. 1), shown in partial longitudinal cross-section. The outer shaft 20 includes a liner 32 defining an outer shaft lumen 34. The liner 32 may be formed of any suitable polymer. In some instances, a low coefficient of friction polymer may be used. Suitable polymers include polyurethane and perfluoro materials such as PTFE (polytetrafluoroethylene), better known as TEFLON®. The inner shaft 24 can be seen as disposed within the outer shaft lumen 34. In some instances, such as in the illustrated embodiment, the outer shaft 20 includes a micromachined region 36 that is disposed within a distal region 38 of the outer shaft 20.

In some instances, the micromachined region 36 may provide the distal region 38 of the outer shaft 20 with additional flexibility while retaining tensile strength. The micromachined region 36 can be seen as including a plurality of voids 40 defining a plurality of rings 42. While not illustrated in this view, each of the plurality of voids 40 are disposed at least substantially circumferentially about the outer shaft 20, but extend only partially about the circumference of the outer shaft 20. In some instances, each of the plurality of voids 40 may extend about half way around the circumference of the outer shaft 20. In some cases, a void 40 may be radially offset from an adjacent void 40.

Similarly, it can be seen that the inner shaft 24 includes a micromachined region 44 that is disposed within a distal region 46 of the inner shaft 24. In some instances, the micromachined region 44 may provide the distal region 46 of the inner shaft 24 with additional flexibility while retaining compressive strength. The micromachined region 44 can be seen as including a plurality of voids 48 defining a plurality of rings 50. While not illustrated in this view, each of the plurality of voids 48 are disposed at least substantially circumferentially about the inner shaft 24 but extend only partially about the circumference thereof. In some instances, each of the plurality of voids 48 may extend about half way around the circumference of the inner shaft 24. In some cases, a void 48 may be radially offset from an adjacent void 48.

The inner shaft 24 has a distal end 52. A stent 54 may be disposed within the outer shaft lumen 34 such that a proximal end 56 of the stent 54 is in proximity to the distal end 52 of the inner shaft 24. In some instances, relative proximal movement of the outer shaft 20 with respect to the inner shaft 24 permits the stent 54 to expand as the outer shaft 20 no longer constrains the stent 54. The distal end 52 of the inner shaft 24 may hold the stent 54 in a stationary position while the outer shaft 20 is withdrawn. In particular embodiments, the stent 54 is a self-expanding stent that will expand once no longer constrained.

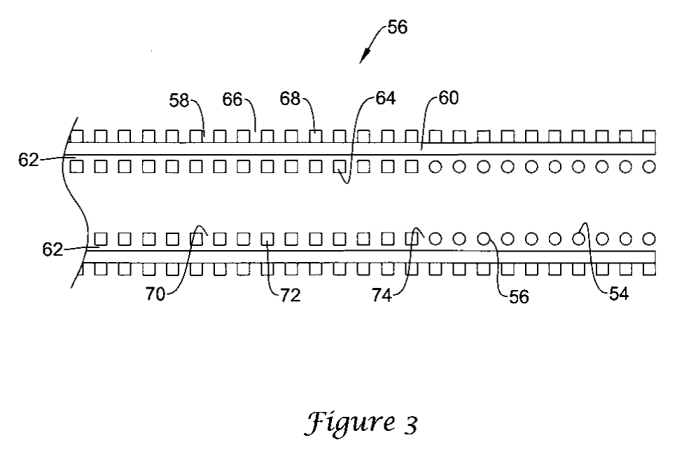

FIG. 3 illustrates a distal portion of a stent delivery catheter 56 in accordance with an embodiment of the invention, shown in partial longitudinal cross-section. The stent delivery catheter 56 includes an outer shaft 58 and a liner 60 defining an outer shaft lumen 62. A inner shaft 64 can be seen as disposed within the outer shaft lumen 62.

In the illustrated embodiment, at least the distal portion of the outer shaft 58 is micromachined to provide the outer shaft 58 with additional flexibility while retaining tensile strength. The outer shaft 58, as a result of being micromachined, includes a plurality of voids 66 that, in concert, define a plurality of rings 68. While not illustrated in this view, each of the plurality of voids 68 are disposed at least substantially circumferentially about the outer shaft 58, but extend only partially about the circumference of the outer shaft 58. In some instances, each of the plurality of voids 68 may extend about half way around the circumference of the outer shaft 58. In some cases, a void 68 may be radially offset from an adjacent void 68.

Similarly, it can be seen that the inner shaft 64 is micromachined in order to provide the inner shaft 64 with additional flexibility while retaining compressive strength. The inner shaft 64 includes, as a result of being micromachined, a plurality of voids 70 defining, in concert, a plurality of rings 72.

While not illustrated in this view, each of the plurality of voids 70 are disposed at least substantially circumferentially about the inner shaft 64 but extend only partially about the circumference thereof. In some instances, each of the plurality of voids 70 may extend about half way around the circumference of the inner shaft 64. In some cases, a void 70 may be radially offset from an adjacent void 70.

The inner shaft 64 has a distal end 74 such that a stent 54 may be disposed within the outer shaft lumen 62 such that a proximal end 56 of the stent 54 is in proximity to the distal end 74 of the inner shaft 54. In some instances, relative proximal movement of the outer shaft 58 with respect to the inner shaft 64 permits the stent 54 to expand as the outer shaft 58 no longer constrains the stent 54. The inner shaft 64 may hold the stent 54 in a stationary position while the outer shaft 58 is withdrawn. In particular embodiments, the stent 54 is a self-expanding stent that will expand once no longer constrained.

The outer shaft 58 and the inner shaft 64 may be formed of any suitable materials such as those discussed with respect to FIG. 2. In particular, the outer shaft 58 and the inner shaft 64 may each be made from or include one or more micromachined hypotubes made of any suitable material such as stainless steel or nitinol.

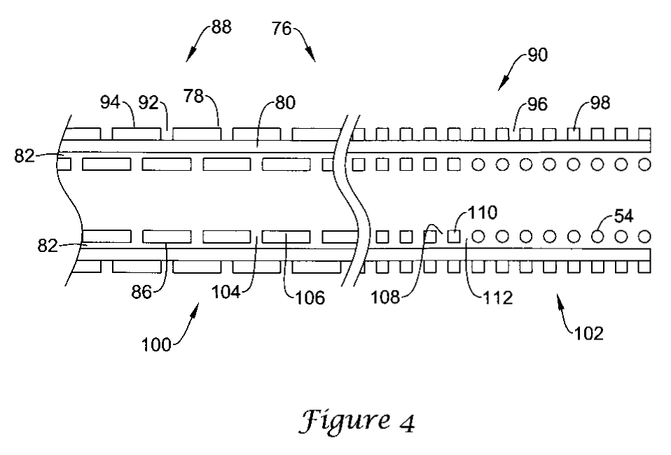

FIG. 4 illustrates a distal portion of a stent delivery catheter 76 in accordance with an embodiment of the invention, shown in partial longitudinal cross-section. The stent delivery catheter 76 includes an outer shaft 78 and a liner 80 defining an outer shaft lumen 82. The liner 80 may be formed of any suitable material, as discussed above. A inner shaft 86 can be seen as disposed within the outer shaft lumen 82. The outer shaft 78 includes a proximal portion 88 and a distal portion 90. While each of proximal portion 88 and distal portion 90 can be seen as including micromachining, the relative spacing differs.

In particular, the proximal portion 88 includes a plurality of voids 92 that define, in concert, a plurality of rings 94. The rings 94 can be seen as relatively wider than the voids 92. As a result, the proximal portion 88 gains some flexibility without significantly sacrificing tensile strength. The distal portion 90 includes a plurality of voids 96 that define, in concert, a plurality of rings 98. Particularly in comparison to the proximal portion 88, the rings 98 are closer in width to the voids 96. As a result, the distal portion 90 gains additional flexibility.

While not illustrated in this view, each of the plurality of voids 92 and 96 are disposed at least substantially circumferentially about the outer shaft 78, but extend only partially about the circumference of the outer shaft 78. In some instances, each of the plurality of voids 92 and 96 may extend about half way around the circumference of the outer shaft 78.

Similarly, it can be seen that the inner shaft 86 includes a proximal portion 100 and a distal portion 102. While each of proximal portion 100 and distal portion 102 can be seen as including micromachining, the relative spacing differs. In particular, the proximal portion 100 includes a plurality of voids 104 that define, in concert, a plurality of rings 106. The rings 106 can be seen as relatively wider than the voids 104. As a result, the proximal portion 100 gains some flexibility without significantly sacrificing tensile strength. The distal portion 102 includes a plurality of voids 108 that define, in concert, a plurality of rings 110. Particularly in comparison to the proximal portion 100, the rings 110 are closer in width to the voids 108. As a result, the distal portion 102 gains additional flexibility.

While not illustrated in this view, each of the plurality of voids 104 and 108 are disposed at least substantially circumferentially about the inner shaft 86, but extend only partially about the circumference of the inner shaft 86. In some instances, each of the plurality of voids 104 and 108 may extend about half way around the circumference of the inner shaft 86.

The inner shaft 86 has a distal end 112 such that a stent 54 may be disposed within the outer shaft lumen 82 such that a proximal end 56 of the stent 54 is in proximity to the distal end 112 of the inner shaft 86.

In some instances, relative proximal movement of the outer shaft 78 with respect to the inner shaft 86 permits the stent 54 to expand as the outer shaft 78 no longer constrains the stent 54. The distal end 112 of the inner shaft 86 may hold the stent 54 in a stationary position while the outer shaft 78 is withdrawn. In particular embodiments, the stent 54 is a self-expanding stent that will expand once no longer constrained.

The outer shaft 78 and the inner shaft 86 may be formed of any suitable materials such as those discussed with respect to FIG. 2. In particular, the outer shaft 78 and the inner shaft 86 may each be made from or include one or more micromachined hypotubes made of any suitable material such as stainless steel or nitinol.

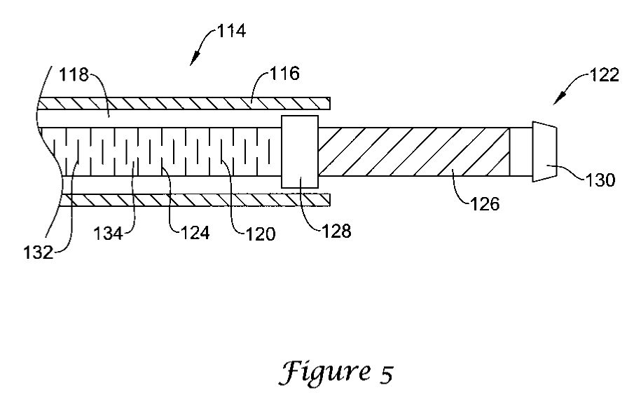

FIGS. 5 through 9 illustrate another particular embodiment of the invention. FIG. 5 shows a distal portion of a stent delivery catheter 114 that includes an outer shaft 116 forming an outer shaft lumen 118 and an inner shaft 120 disposed within the outer shaft lumen 118. The stent delivery catheter 114 has a distal end 122. The outer shaft 116 may be formed of any suitable material. In some instances, the outer shaft 116 may be a metallic micromachined hypotube as discussed with respect to the earlier Figures. The outer shaft 116 may be formed of any suitable polymeric material and may or may not include a lubricious inner coating or liner (not illustrated.

The inner shaft 120 includes a micromachined proximal section 124 and a coil distal section 126. The micromachined proximal section 124 can be formed of any suitable metallic or polymeric material and may include voids 132 defining rings 134. The coil distal section 126 can be formed of any suitable metallic or polymeric material and otherwise is of conventional design. In some instances, the micromachined proximal section 124 may be formed from a first tube while the coil distal section 126 may be formed from a second tube. In particular instances, the proximal section 124 and the distal section 126 are formed from a single tube.

A stent stop 128 is mounted onto the inner shaft 120 proximate the union between the micromachined proximal section 124 and the coil distal section 126. The stent stop 128 may be formed of any suitable metallic or polymeric material and may be attached to the inner shaft 120 using any suitable technique. A distal tip 130 is secured to the distal end 122 of the inner shaft 120.

The inner shaft 120 may be formed of any suitable metallic or polymeric material, and may have a variety of cross-sectional shapes. The inner shaft 120 may define an inner lumen, or may in some configurations be solid in radial cross-section. An inner lumen within the inner shaft 120 may be used to accommodate a guidewire or similar device. If the inner shaft 120 is solid, the stent delivery catheter 114 may include other structure for accommodating a guidewire, such as rapid exchange capability.

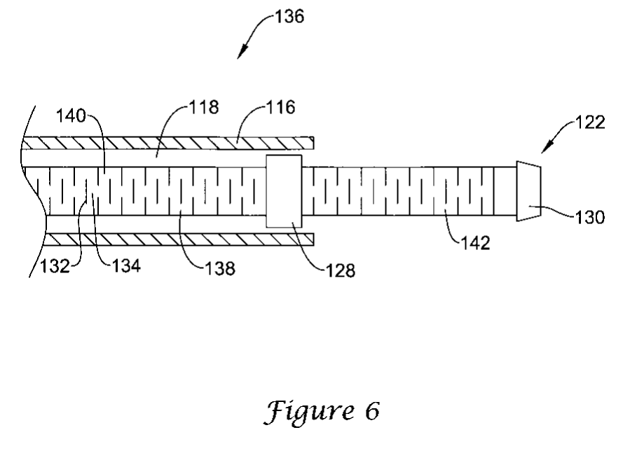

FIG. 6 shows a distal portion of a stent delivery catheter 136 that includes an outer shaft 116 forming an outer shaft lumen 118 and an inner shaft 138 disposed within the outer shaft lumen 118, as discussed with respect to FIG. 5. The inner shaft 138 includes a micromachined proximal section 140 and a micromachined distal section 142. The micromachined proximal section 140 can be formed of any suitable metallic or polymeric material and may include voids 132 defining rings 134 as discussed with respect to the inner shaft 120 of FIG. 5.

A stent stop 128 is mounted onto the inner shaft 138 proximate the union between the micromachined proximal section 140 and the micromachined distal section 142. The stent stop 128 may be formed of any suitable metallic or polymeric material and may be attached to the inner shaft 120 using any suitable technique. A distal tip 130 is secured to the distal end 122 of the inner shaft 138.

The inner shaft 138 may be formed of any suitable metallic or polymeric material, and may have a variety of cross-sectional shapes. The inner shaft 138 may define an inner lumen, or may in some configurations be solid in radial cross-section. An inner lumen within the inner shaft 138 may be used to accommodate a guidewire or similar device. If the inner shaft 138 is solid, the stent delivery catheter 136 may include other structure for accommodating a guidewire, such as rapid exchange capability.

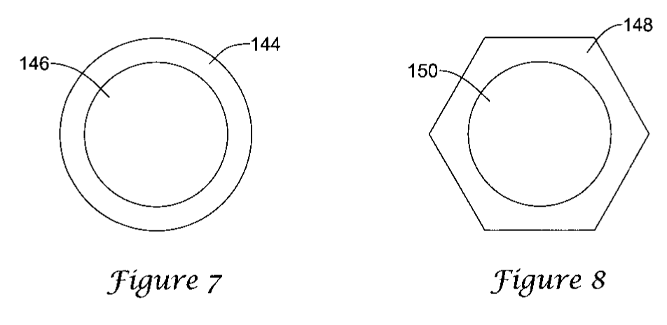

FIGS. 7 and 8 are diagrammatic cross-sections of a portion of an inner shaft in accordance with an embodiment of the invention. In FIG. 7, an inner shaft 144 defines an inner shaft lumen 146. The inner shaft 144 and the inner shaft lumen 146 both have circular or nearly circular radial cross-sectional profiles. In FIG. 8, however, an inner shaft 148 defines an inner shaft lumen 150. While the inner shaft lumen 150 has a circular or nearly circular radial cross-sectional profile, the inner shaft 148 has a polygonal radial cross-sectional profile. Differing geometries may provide advantages in strength versus flexibility, as well as possibly reducing frictional forces.

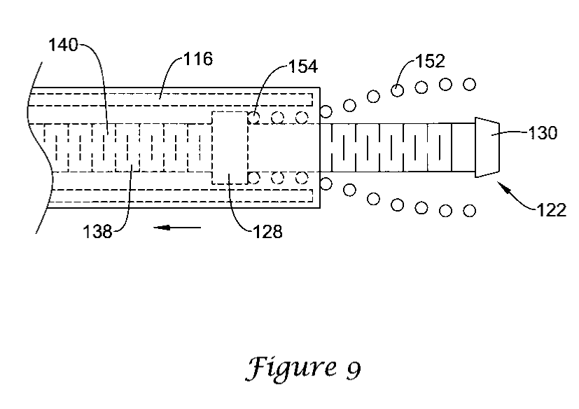

Function of the stent delivery catheter 136 (seen in FIG. 6) is demonstrated in FIG. 9. A stent 152, which may be a self-expanding stent, has been positioned within the outer shaft 116 and is deployed on the micromachined distal section 142. The stent 152 can be considered as having a length that is equal to or less than a length between the stent stop 128 and the distal tip 130. The stent 152 has a proximal end 154 that rests against the stent stop 128 such that the stent stop 128 hinders proximal movement of the stent 152 as the outer shaft 116 is moved proximally, thereby exposing and thus deploying the stent 152.

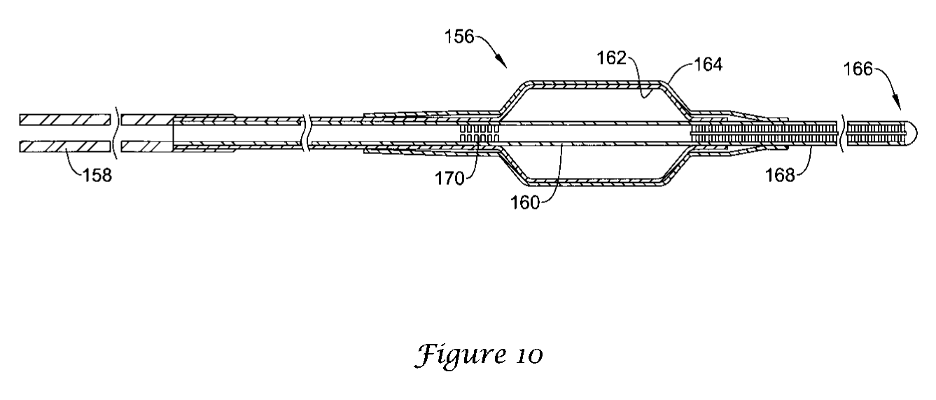

FIG. 10 illustrates a stent delivery catheter 156 that includes a proximal tube 158 and a micromachined distal tube 160. In some instances, the proximal tube 158 may also be micromachined. The micromachined distal tube 160 may be considered as having a plurality of rings and voids, as discussed previously. The distal tube 160 is attached to and may extend partially into the proximal tube 158. In some instances, the proximal tube 158 may be formed from a first tube while the micromachined distal tube 160 may be formed by processing a second tube. In particular instances, the proximal tube 158 and the micromachined distal tube 160 are formed from a single tube.

An inflatable balloon 162 is disposed over the micromachined distal tube 160 and in fact the inflatable balloon 162 extends proximally to cover all of the micromachined distal tube 160. In some instances, such as that illustrated, an elastomeric sleeve 164 may be disposed over the inflatable balloon 162 to control outward expansion of the inflatable balloon 162 when an inflating fluid such as saline or contrast medium is provided into the inflatable balloon 162. The elastomeric sleeve 164 may be excluded, if desired.

The stent delivery catheter 156 has a distal end 166 and may include a marker coil 168 disposed between the distal end 166 and the inflatable balloon 162, inside of the micromachined distal tube 160. A second marker coil 170 may be disposed in or on the micromachined distal tube 160 within the inflatable balloon 162.

The proximal tube 158 may be formed of any suitable material, such as a nickel-titanium alloy, titanium or perhaps a polymeric material. If the proximal tube 158 is formed of a polymer, a reinforcing braid (not illustrated) may be incorporated. In a particular embodiment, the proximal tube 158 includes or is formed of stainless steel. The micromachined distal tube 160 may be formed of any suitable material such as stainless steel or titanium, but in particular instances may be formed of a nickel-titanium alloy such as nitinol.

Any suitable polymeric materials may be used for the inflatable balloon 162 and the elastomeric sleeve 164. Examples include polyethylene, HYTREL®, polyester, polyurethane and PEBAX®. In particular, the inflatable balloon 162 may include or be formed from NYLON 12®. The marker coils 168 and 170 may be formed of any suitably radiopaque material. Examples include platinum and platinum alloys, and gold and gold alloys. In particular, the marker coils 168 and 170 may be formed of a platinum-tungsten alloy.

While not expressly illustrated, the stent delivery catheter 156 may include one or more polymeric sleeves, layers or coatings exterior to the proximal tube 158. Such a sleeve, layer or coating may include or be formed of any suitable material.

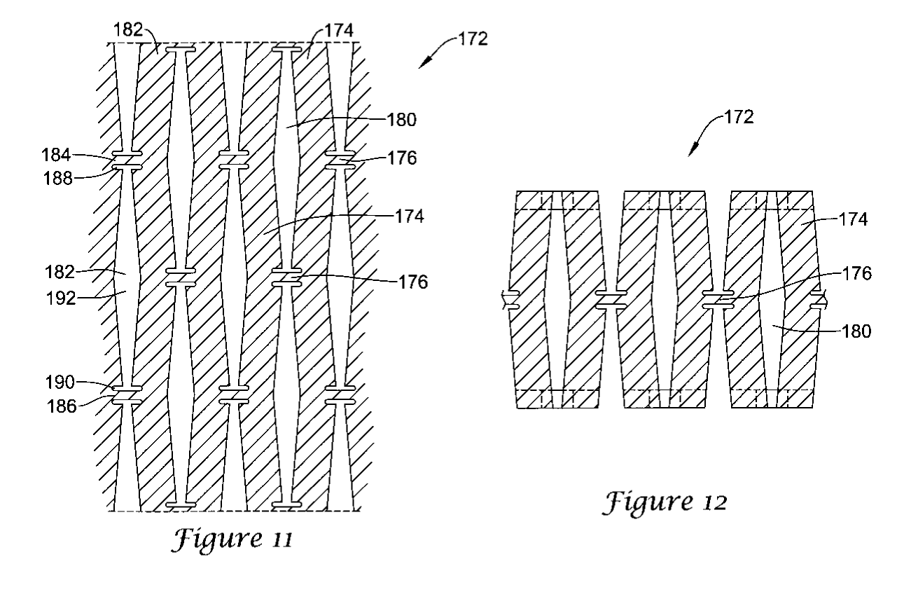

FIGS. 11 and 12 illustrate a particular cutting pattern for forming a micromachined hypotube, such as those discussed with respect to the previous Figures. FIG. 11 diagrammatically shows the cutting pattern as if a hypotube has been un-rolled, while FIG. 12 demonstrates the cutting pattern on an intact hypotube.

A hypotube 172 includes a number of rings 174 that are sized and configured to provide tensile strength as well as a number of beams 176 that are interspersed between adjacent rings 174. The beams 176 are sized and configured to provide flexibility to the hypotube 172. A number of voids 180 extend from an interior surface (not seen) to an exterior surface 182 and, in combination, define the rings 174 and the beams 176. In some instances, the voids 180 can be considered as permitting the hypotube 172 to bend without permitting adjacent rings 174 to contact each other.

In particular, a void 182 (of the number of voids 180) can be seen as extending circumferentially about the hypotube 172 from a first beam 184 to a second beam 186. The void 182 includes a first widened portion 188 that is proximate the first beam 184 and a second widened portion 190 that is proximate the second beam 186. It can be seen that the first widened portion 188 functionally lengthens the adjoining first beam 184 and that the second widened portion 190 functionally lengthens the adjoining second beam 186. Thus, the first widened portion 188 and the second widened portion 190 serve to increase the flexibility of the hypotube 172.

The void 182 further includes an intermediate portion 192 that is disposed between the first widened portion 188 and the second widened portion 190. The intermediate portion 192 includes an intermediate diameter that is greater than a diameter of the intermediate portion 192 proximate either the first widened portion 188 or the second widened portion 190. Moreover, it can be seen that each of the rings 174 are larger in axial dimension than each of the voids 180.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

We claim:

1. A stent delivery catheter having a distal region and a proximal region, the distal region defining a distal end, the stent delivery catheter comprising:
an outer shaft defining an outer shaft lumen extending therethrough, the outer shaft extending from the proximal region to the distal region; and
a inner shaft disposed within the outer shaft lumen;
wherein at least one of the outer shaft and the inner shaft comprise a micromachined portion thereof.

2. The stent delivery catheter of claim 1, wherein the outer shaft includes a micromachined distal region.

3. The stent delivery catheter of claim 2, wherein the outer shaft further includes a micromachined proximal region.

4. The stent delivery catheter of claim 3, wherein the micromachined distal region comprises a plurality of slots having a first inter-slot spacing, the micromachined proximal region comprises a plurality of slots having a second inter-slot spacing, and the first inter-slot spacing is less than the second inter-slot spacing.

5. The stent delivery catheter of claim 1, wherein the outer shaft comprises a metallic hypotube.

6. The stent delivery catheter of claim 1, wherein the outer shaft comprises a polymer shaft.

7. The stent delivery catheter of claim 1, wherein the outer shaft further comprises a liner disposed within the outer shaft lumen.

8. The stent delivery catheter of claim 1, wherein the inner shaft includes a micromachined distal region.

9. The stent delivery catheter of claim 8, wherein the inner shaft further includes a micromachined proximal region.

10. The stent delivery catheter of claim 1, wherein the inner shaft comprises a metallic hypotube.

11. The stent delivery catheter of claim 1, wherein the inner shaft comprises a polymer.

12. The stent delivery catheter of claim 11, wherein the inner shaft comprises a polymeric sheath having a circular radial cross-section.

13. The stent delivery catheter of claim 11, wherein the inner shaft comprises a polymeric sheath having a polygonal radial cross-section.

14. The stent delivery catheter of claim 1, wherein the inner shaft and the outer shaft both extend distally to the distal end of the catheter, the inner shaft comprising a stent stop positioned to accommodate a self-expanding stent disposed between the inner shaft and the outer shaft.

15. The stent delivery catheter of claim 14, wherein the stent stop is configured such that relative axial movement between the outer shaft and the inner shaft causes the self-expanding stent to contact the stent stop.

16. The stent delivery catheter of claim 14, wherein a portion of the inner shaft proximal of the stent stop comprises a micromachined tube and a portion of the inner shaft distal of the stent stop comprises a coil.

17. The stent delivery catheter of claim 1, wherein the outer shaft extends to the distal end of the catheter, and the inner shaft has a distal end positioned proximally of the distal end of the catheter such that a self-expanding stent can be accommodated within the outer shaft lumen distally of the inner shaft.

18. The stent delivery catheter of claim 17, wherein the inner shaft is configured such that the distal end of the inner shaft contacts the self-expanding stent.

19. A catheter having a distal region and a proximal region, the distal region defining a distal end, the catheter comprising:
a micromachined hypotube having a proximal end and a distal end, the micromachined hypotube extending distally to the distal end of the catheter;
a proximal tube extending proximally from the proximal end of the micromachined hypotube; and
an inflatable balloon disposed over the micromachined hypotube, the inflatable balloon defining a balloon interior.

20. The catheter of claim 19, further comprising an elastomeric sleeve disposed over the inflatable balloon.

21. The catheter of claim 19, wherein the inflatable balloon comprises a proximal balloon shaft that extends proximally to the proximal end of the micromachined hypotube such that a portion of the micromachined hypotube proximal of the inflatable balloon is sealed.

22. The catheter of claim 19, further comprising an atraumatic tip disposed at the distal end of the catheter.

23. The catheter of claim 19, further comprising at least one marker band disposed proximate the micromachined hypotube.

24. The catheter of claim 19, further comprising a balloon-inflatable stent disposed over the inflatable balloon.

25. A micromachined hypotube comprising:
an elongate cylindrical shaft having an interior surface and an exterior surface;
a plurality of rings formed within the elongate cylindrical shaft, the plurality of rings sized and configured for tensile strength;
a plurality of beams interspersed between adjacent rings, the plurality of beams sized and configured for flexibility; and
a plurality of voids extending between the interior surface and the exterior surface, the plurality of voids defining the plurality of rings and the plurality of beams.

26. The micromachined hypotube of claim 25, wherein the plurality of voids are configured to permit the micromachined hypotube to bend without adjacent rings contacting each other.

27. The micromachined hypotube of claim 25, wherein a first void of the plurality of voids extends circumferentially about the elongate cylindrical shaft between a first beam of the plurality of beams and a second beam of the plurality of beams.

28. The micromachined hypotube of claim 27, wherein the first void comprises a first widened portion proximate the first beam and a second widened portion proximate the second beam.

29. The micromachined hypotube of claim 28, wherein the first widened portion functionally lengthens the adjoining first beam and the second widened portion functionally lengthens the adjoining second beam.

30. The micromachined hypotube of claim 28, wherein the first void comprises an intermediate portion between the first widened portion and the second widened portion, the intermediate portion having an intermediate diameter that is greater than a diameter of the intermediate portion proximate the first widened portion or the second widened portion.

31. The micromachined hypotube of claim 25, wherein each of the plurality of rings are larger in axial dimension than each of the plurality of voids.

32. A stent delivery catheter comprising:
an outer shaft comprising the micromachined hypotube of claim 25; and
a inner shaft disposed within the outer shaft.

33. The stent delivery catheter of claim 32, further comprising a self-expanding stent disposed within the outer shaft.
